(19) United States
(12) Patent Application Publication
(10) Pub. No.: US 2003/0150461 A1
(43) Pub. Date: Aug. 14, 2003

(54) Endotracheal tube with aerosol delivery apparatus

(76) Inventors: Sunil Kumar Dhuper, Old Westbury, NY (US); Sarita Dhuper, Old Westbury, NY (US)

Correspondence Address: SUNIL DHUPER, 47, RED GROUND ROAD, OLD WESTBURY, NY, 11568, US

(21) Appl. No.: 10/072,282

(22) Filed: Feb. 11, 2002

(51) Int. Cl. A61M 16/00

(52) U.S. Cl. 128/207.14; 128/207.15; 128/200.26

(57) ABSTRACT

Current methods of drug administration to the lungs are inefficient. ‘Endotracheal Tube with Aerosol Delivery Apparatus’ is specifically designed for uniform intrapulmonary deposition of aerosolized medication in patients on mechanical ventilation. As opposed to the current methods of drug delivery where aerosol particles are generated at the proximal end of the ET tube, with majority of the particles adhering to the endotracheal tube during delivery, this invention bypasses the endotracheal tube by generating aerosol particles at its distal end. This invention incorporates an external adapter designed to perfectly fit the nozzle of the conventional metered dose inhaler at it's proximal end. From the distal end of this adapter originates a secondary cannula with a pinhole inner diameter that enter the ET tube at its proximal end and continues distally within the wall of the ET tube to terminate as a pinhole opening, where aerosol particles are generated.

### CROSS-REFERENCE TO RELATED APPLICATIONS

| | | | |
|---|---|---|---|
| 3114373 | June, 1962 | Anderson | 604/45 |
| 3173418 | March, 1965 | Baran | 128/207 |
| 3638655 | February, 1972 | Doherty | 125/207 |
| 3788326 | January, 1974 | Jacobs | 128/207 |
| 3981299 | September, 1976 | Murray | 604/43 |
| 4022219 | May, 1977 | Basta | 128/207 |
| 3173418 | May, 1977 | Baran | 128/207 |
| 4119101 | October, 1978 | Igich | 128/202 |
| 4214592 | July, 1980 | Imbruce | 600/556 |
| 4327720 | May, 1982 | Brunson et al | 128/207 |
| 4417576 | November, 1983 | Baran | 128/207 |
| D272094 | January, 1984 | Wolf et al | D24/110 |
| 4453545 | June, 1984 | Inoue | 128/207 |
| 4569344 | February, 1986 | Palmer | 128/207 |
| 4584998 | April, 1986 | McGrail | 128/207 |
| 4607635 | August, 1986 | Heyden | 128/207 |
| 4637389 | January, 1987 | Heyden | 128/207 |
| 4674495 | June, 1987 | On | 128/207 |
| 4669463 | June, 1987 | McConnell | 128/207 |
| 4693243 | September, 1987 | Buras | 128/207 |
| D307183 | April, 1990 | Kalayjian | D24/110 |
| 4955375 | September, 1990 | Martinez | 128/207 |
| 4977894 | December, 1990 | Davies | 128/207 |
| 5029580 | July, 1991 | Radford | 128/207 |
| D328244 | July, 1992 | Hamilton et al | D9/338 |
| 5146916 | September, 1992 | Catalani | 128/207 |
| D335175 | April, 1993 | Sladek | D241110 |
| 5513630 | May, 1996 | Century | 128/203 |
| 5542412 | August, 1996 | Century | 128/203 |
| 5570686 | November, 1996 | Century | 128/203 |
| 579758J | January, 1997 | Century | 441/60 |
| 5594987 | January, 1997 | Century | 29/890 |
| 5606789 | March, 1997 | Century | 29/281 |

### STATEMENT REGARDING FEDERALLY SPONSORED RESEARCH OR DEVELOPMENT

Not Applicable

### REFERENCE TO A SEQUENCE LISTING, A TABLE, OR A COMPUTER PROGRAM LISTING COMPACT DISK APPENDIX

Not Applicable

### BACKGROUND OF THE INVENTION

The present invention relates to medical-surgical devices for intubation i.e. endotracheal tube (ET tube) intended for tracheal insertion in patients requiring mechanical ventilation. This tube is specifically designed for effective intrapulmonary deposition of aerosol particles-quantitatively as well as qualitatively (uniform distribution in tracheobronchial tree) in patients on mechanical ventilation via endotracheal tube. Many therapeutic substances can be utilized through this route, to name a few, bronchodilators, anti-inflammatory agents like steroids, antibiotics, anticholinergics, heparin, surfactant, antiproteases, gene transfer products, etc.

The advantages of intrapulmonary drug delivery as opposed to systemic administration are well known. Multiple medications as outlined above readily lend themselves for pulmonary administration. Current methods of drug administration to the lungs are inefficient. Not only are they limited in delivery of quantitatively significant amount of medication to the lungs, but they have also failed qualitatively to achieve uniform intrapulmonary distribution.

There are two methods currently available for intrapulmonary drug delivery.

(I) Liquid bolus: The medication is instilled in the form of liquid bolus via a bronchoscope or through an endotracheal (ET) tube. Not only is the distribution by this method non-uniform but there is also a significant risk of inducing respiratory distress and hypoxemia.

(II) Aerosol Inhalation: Methods employed use Metered Dose Inhalers (MDI's) with low boiling point propellants such as chlorofluoroalkanes or aerosol particles generated by heat, traditional compressed air nebulizers, or ultrasonic nebulizers. This method, even though it produces a more uniform distribution of aerosol particles compared with liquid bolus method, is limited in quantitatively delivering significant amount of medication to the lungs. Only a small fraction of the medication reaches the lungs and majority of the aerosol particles either adhere to the nasal passages and oropharynx or are exhaled out. Efficiency of aerosol delivery drops down even further in patients who are intubated and require mechanical ventilation. Beck et al found that inhalation of nebulized material through an endotracheal tube resulted in deposition of only 1.87% of the delivered particles to the lungs. Methods employing a combined ventilator dispenser and adapter (U.S. Pat. No. 335,175) with MDI's have revealed equally poor results because much of the aerosol particles adhere to the ET tube and the inspiratory limb of the corrugated plastic tube.

Investigators over the years have devised numerous endotracheal tubes for intrapulmonary drug delivery. Most designs of endotracheal tubes so far have only addressed the issue of drug delivery in the form of liquid bolus by incorporating drug irrigation devices in the traditional ET tube either in the form of secondary canalization with multiple micrometric openings (U.S. Pat. No. 5,146,936) or with some such modification of the original design.

Generation and delivery of aerosol particles with small mid-mean diameter, which is critical for uniform deposition in the tracheobronchial tree especially to reach the small airways, has not been addressed by any of the currently existing endotracheal tubes incorporating drug irrigation devices. Recently one of the investigators invented a delivery device for intratracheal administration of drug in aerosol form called ‘Penn Century Intracheal Aerosolizer (Microsprayer)’ [U.S. Pat. Nos. 5,579,758, 5,594,987, 5,606,789, 5,513,630, 5,542,412, 5,570,686]. This device is not related to our field of invention i.e. medical surgical devices for intubation. The clinical utility of this device in humans at this time is extremely limited because of its high cost and need for sterilization after every use and as such it is solely being used as a research tool.

### BRIEF SUMMARY OF THE INVENTION

### Objects of Invention

The main object of the present invention is to provide a modified ET tube that serves the following purposes:

Aerosol drug delivery to tracheobronchial tree.

Generation and delivery of aerosol particles at the distal end of the ET tube with mid mean diameter that will allow uniform distribution throughout the tracheobronchial tree.

Generation and delivery of aerosol particles at the distal end of the ET tube so as to quantitatively deliver significant fraction of the generated aerosol particles to the tracheobronchial tree without adherence to the ET tube. This also implies cost effectiveness by preventing waste of medication.

Simple inexpensive method of intrapulmonary drug delivery

To achieve all the previous mentioned objects without interfering with the function of the ET tube.

To achieve the above objectives through a device that does not impede intubation or in anyway make it more complicated for the operator, or more traumatic to the patient.

The defined objects are obtained through our invention i.e. the ET tube that incorporates the following new features:

External Medicament Dispenser with Adapter (MDA)—An external MDA is attached to the ET tube at its proximal end. The adapter is specifically designed such that the outer circumference of the cylindrical nozzle located at the end of a conventional metered dose inhaler (MDI) cannister perfectly fits into the inner circumference of the cylindrical frame work of MDA. Aerosol particles are generated when the MDI valve is actuated. The use of an MDI for intrapulmonary delivery of various medications is well known. An MDI consists of a pressurized cannister containing powdered medication with a low boiling point propellant maintained in liquid state. When the valve of the MDI is activated, the propellant is released and forces medicament from the nozzle of the cannister along with propellant. Since the essence of this invention disclosed herein does not relate specifically to the structure of an MDI device, the details of this construction will not be discussed herein. Means of making and using MDI are well known to those skilled in the art. The adapter tapers at its distal end to reach an inner diameter (ID) of a pinhole (range 0.1 mm-1.0 mm). The distal tip of the adapter marks the origin of a secondary cannula. This feature of our invention differentiates it from most of the other existing adapters that have a pinhole opening on one of the sides, a few millimeters higher than the distal end of the adapter which is generally closed.

Secondary cannula (semi-flexible part)—Originating from the distal end of the MDA is a semi-flexible cannula. The ID of the cannula same as the ID of the pinhole opening at the distal tip of the MDA. The cannula can vary in length (generally<10 cm) depending on the size of the ET tube. The semi-flexible cannula enters into the wall of the ET tube through an opening on the outer surface of the ET tube. The point of entry of the secondary cannula is on the outer lateral surface of the ET tube compared with the primary cannula for the balloon cuff which enters on the convex surface, so as not to make intubation complicated for the operator. Note that the semi-flexible cannula could be made semi-rigid by increasing the thickness of its wall and using a stiffer plastic material without changing the ID.

Secondary cannula (rigid part)—The secondary cannula continues distally within the wall of the ET tube to terminate as a pinhole opening at the tip of the ET tube. The ID of the rigid part of the secondary cannula is kept the same as the ID of flexible part. The track of the secondary canalization within the wall of the ET tube is from the outer surface to the inner surface. The narrow lumen of the secondary cannula allows the particles generated by MDI to reach the distal tip of the ET tube in aerosol form without adherence to the inner surface of the ET tube.

### BRIEF DESCRIPTION OF THE SEVERAL VIEWS OF THE DRAWING

Further features of the present invention will become apparent in the accompanying drawings as well as the detailed description of the preferred embodiments.

FIG. 1 show the longitudinal length of the ET tube incorporating all the features described in the summary i.e. MDA, secondary cannula—the semi flexible part outside the main body of the ET tube and the rigid part within the wall of the ET tube.

FIG. 2 shows the longitudinal length of the ET tube associated with alternative embodiment of secondary cannulation.

FIGS. 3a, 3b, 3c and 3d show a cross section of the ET tube (FIG. 1) taken at four levels.

FIG. 3a—cross section at level 1 (L-L₁)

FIG. 3b—cross section at level 2 (L-L₂)

FIG. 3c—cross section at level 3 (L-L₃)

FIG. 3d—cross section at level 4 (L-L₄)

FIGS. 4a, 4b, 4c and 4d show a cross section of the ET tube (FIG. 2) taken at four levels.

FIG. 4a cross section at level 5 (L-L₅)

FIG. 4b cross section at level 6 (L-L₆)

FIG. 4c cross section at level 7 (L-L₇)

FIG. 4d cross section at level 8 (L-L₈)

FIGS. 5a, 5b, 5c, and 5d show the perspective views of the MDA.

FIG. 5a shows the oblique view of the MDA

FIG. 5b represents the front and rear elevational view

FIG. 5c represents the left and right elevational view

FIG. 5d represents the cross section (top and bottom views)

### DETAILED DESCRIPTION OF THE INVENTION

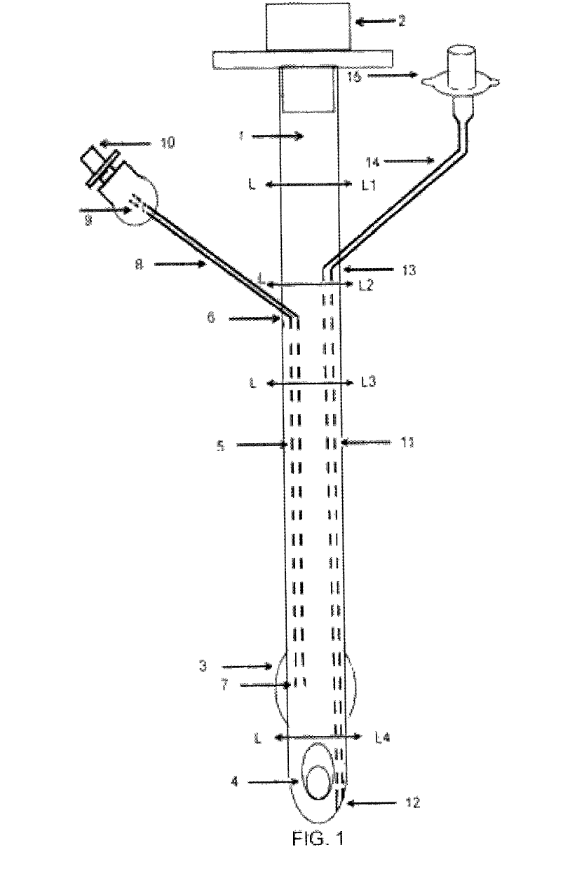

FIG. 1 shows the longitudinal length of an ET tube which is composed of an elongated hollow tube (1) approximately 34 cm long made of plastic material. The internal diameter of the tube could vary from 2.5 mm to 10 mm and the external diameter could vary from 3.5 mm to 13 mm. The thickness of the wall of the tube could vary from 0.5 mm to 2.0 mm. The tube is a flexible elongated conduit with a concave surface on one side and a convex surface on the opposite side. It's proximal is end connected to an adapter (2) which enables it to be connected to an elongated tube of a mechanical ventilator. The distal end has a 4 cm expandable cuff (3) starting approximately 4 cm from the distal tip and ending approximately 8 cm from the distal tip. In the distal 4 cm of the ET tube, between the expandable cuff and the distal tip of the ET tube, there is a pair of oval holes (4) one each on the opposite surfaces of the tube facing each other. The size of the holes can vary between 5 mm and 1 cm. A small tube i.e. primary cannulation (5) of approximately 1 mm diameter runs within the wall on the convex side of the tube(s) and is connected to the expandable cuff for inflation and deflation by terminating on the outer surface of the ET tube as a 1 mm hole (7). This tube alternatively can be attached on the outer surface of the tube on the convex side. This primary cannula has a proximal flexible part (8) (approximately 18 cm from the distal tip of the ET tube) which continues outside the main tubular structure of the ET tube (1). The flexible part starts at approximately 18 cm from the distal tip of the ET tube and continues proximally for a few centimeters to terminate into a cuff inflation indicator (9) and adapter (10) for a syringe. The connection between the flexible and rigid part of the primary cannula is through an opening on the outer surface of the ET tube (6) which is also 1 mm in ID. On the lateral surface of the ET tube starting at the same level as the canalization for the inflation of the balloon (6) or higher level (13) there originates another secondary cannula (11) on the outer surface of the ET tube and continues within the wall of the ET tube. The ID of this secondary canalization can vary from 0.1 mm to 1.0 mm in size. This secondary canalization continues distally beyond the balloon to terminate as a pinhole opening (12) at the distal tip of the ET tube. Of note is that the course of the secondary canalization within the wall of ET tube is from the outer surface to the inner surface and it terminates on the inner surface at the distal tip of the ET tube. The canalization is an extension of a semi-flexible proximal cannula (14) which is on the outside of the main tubular structure of the ET tube (1) without adhering to it just like the flexible part of the primary cannula (8). The semi-flexible cannula (14) makes a connection with the secondary canalization (11) through an opening (13) on the outer surface of the ET tube. The flexible cannula is an extension of medicament dispenser and adapter (15) that the ET tube is equipped with at its proximal end. The adapter is designed to fit the nozzle of a metered dose inhaler (MDI) cannister.

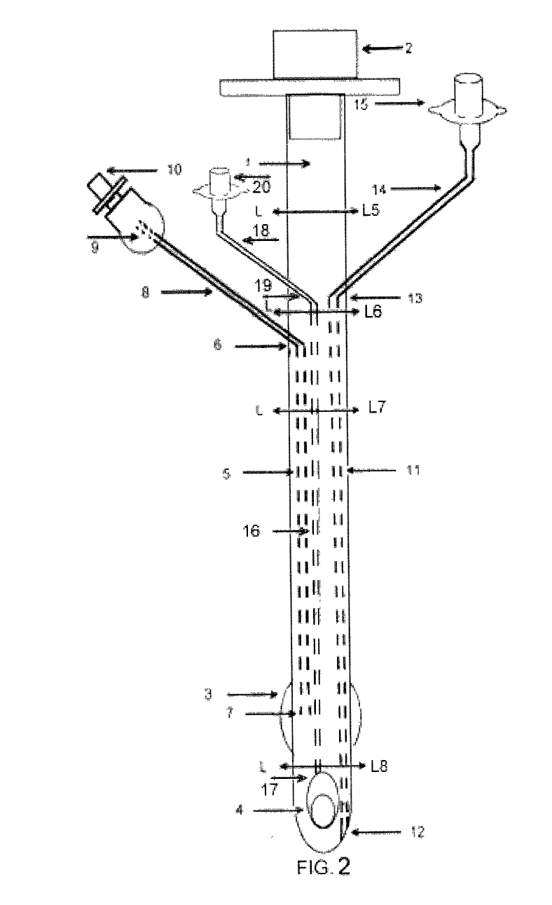

FIG. 2 shows the longitudinal view of the ET tube associated with alternative embodiments of secondary canalization. FIG. 2 is the same as FIG. 1 but with an additional secondary cannula (16) running an identical track on the opposite lateral surface of the ET tube as the secondary cannula (11). The additional secondary cannula (16) continues distally like secondary cannula (11) to terminate as a pinhole opening at its distal tip (17). The secondary cannula (16) is an extension of a semi-flexible proximal cannula (18); the two making a connection through an opening (19) on the outer lateral surface of the ET tube. The flexible cannula is an extension of the distal part of the second MDA (20) designed to fit the nozzle of MDI cannister. Note that the flexible part (18) can also be made semi-rigid while keeping all other parts and connections the same. In this respect the length of the cannula outside the main tubular structure could be shortened and the secondary cannula in the wall could be elongated.

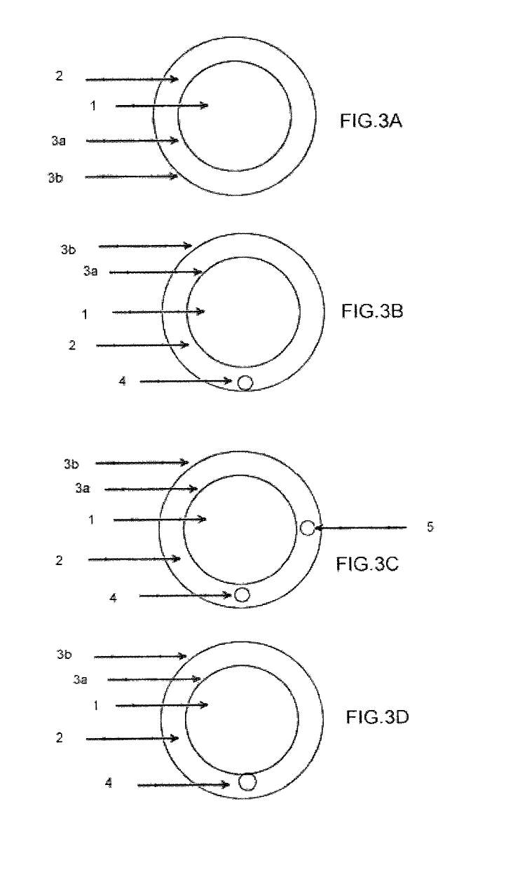

FIGS. 3a, 3b, 3c and 3d show details of 4 cross sections at 4 levels of the ET tube as shown in FIG. 1 FIG. 3a is the cross section at level L-L1 which shows the hollow tube (1), the wall of the tube (2), the inner surface of the wall (3a) and the outer surface of the wall (3b). FIG. 3b is the same as FIG. 3a but with the appearance of an additional secondary canalization (4) starting close to the outer wall (3b). FIG. 3c is the same as FIG. 3b but with a difference in the position of the secondary canalization (4) which is now in the center of the wall 2. Also there is an additional primary cannula (5) on the convex side of the ET tube near the outer wall for inflation and deflation of the balloon cuff. FIG. 3d is the same as FIG. 3c but with the absence of primary cannula (5) as it terminates at a higher level near the expandable balloon. The secondary cannula (4) is closer to the inner wall (3a).

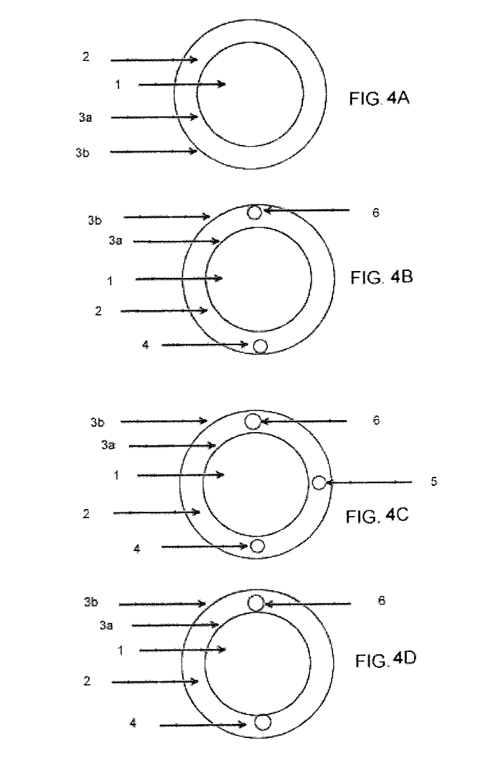

FIGS. 4a, 4b, 4c and 4d show details of 4 cross sections at 4 different levels as seen in FIG. 2 FIG. 4a is the same as FIG. 3a FIG. 4b is the same as FIG. 3b but with an additional secondary canalization (6) on the contralateral side of the secondary canalization (4). FIG. 4c is the same as FIG. 3c but with an additional secondary canalization (6) FIG. 4d is the same as FIG. 3d but with an additional secondary canalization (6)

Note that the tracks of both the secondary canalizations in the wall of the ET tube run from the outer wall (3a) to the inner wall (3b) of the ET tube. The primary canalization (5) stays towards the outer wall (3a) all through it's course in the wall of the ET tube. It is obvious that the shape and location of the primary and secondary cannulations can be different from those illustrated above subject to the purpose of invention. The cannulations can continue on the outer surface or on the inner surface of the ET tube and not in the wall of the ET tube or could alternatively run partly on the inner and/or outer surface and partly within the wall of the ET tube.

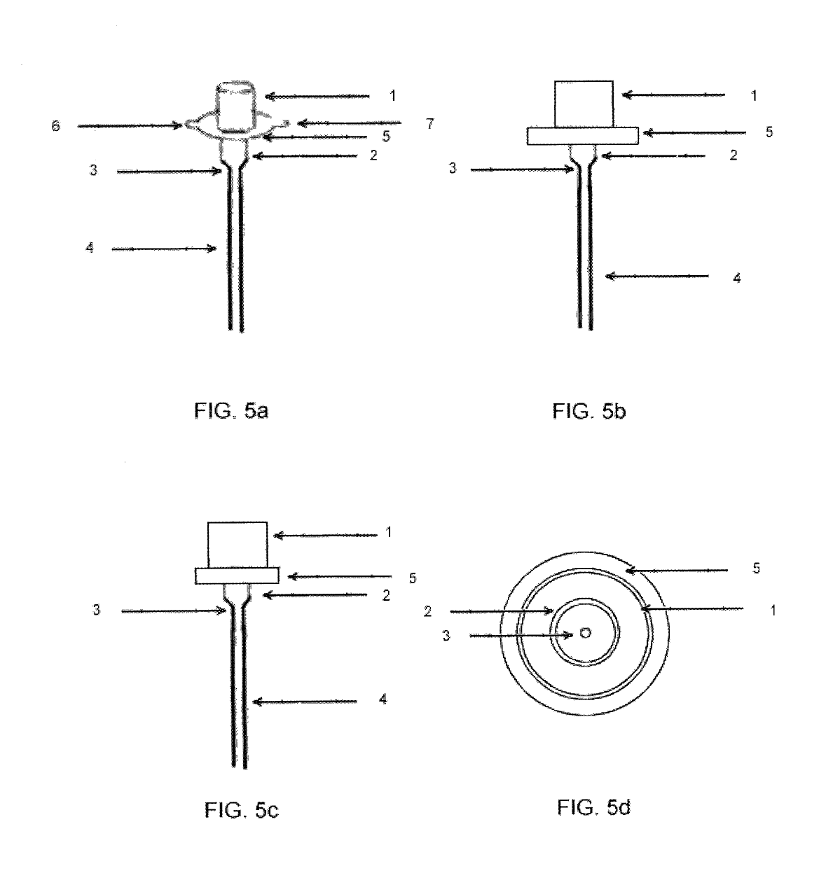

FIG. 5a is the oblique view of the medicament dispenser and adapter in the shape of a cylinder. The inner circumference of the proximal part (1) decreases to terminate as a distal cylindrical part (2). The inner circumference of the distal part (2) is designed to fit the outer circumference of the cylindrical nozzle of the MDI cannister. The distal part (2) of the adapter continues for 1-2 mm distally to reach a pinhole ID (3) which marks the origin of a flexible catheter (4) {also described as (14) and (18) in FIG. 1 and 2 respectively.} Wrapped around the adapter is a central circular plate (5) with two side handles one each for the index finger (6) and the middle finger (7) to press against, when compressing the MDI with the thumb to actuate the valve of the MDI. FIG. 5b represents the front and rear elevational views of MDA described in FIG. 5a. FIG. 5c represents the left and right elevational views of MDA described in FIG. 5a. FIG. 5d represents the cross-section (top and bottom views) of MDA described in FIG. 5a. The wall of the proximal cylindrical part (1), the wall of the distal cylindrical part (2), the pinhole opening of the distal cylindrical part (3) that marks the origin of semi-flexible cannula (described as (4) in FIG. 5a) and the circular plate (5) are described here.

It is noted that the illustration (drawings) and description of the preferred embodiments have been provided merely for the purpose of explanation and although the invention has been described herein with reference to particular means, materials and embodiments, the invention is not intended to be limited to the particulars disclosed herein; rather the invention intends to all functionally equivalent structures, methods and uses such as are within the scope of the appended claims.

We claim:

1 An endotracheal tube
with an expandable balloon cuff at its distal end
with a primary cannula for inflation and deflation of the balloon cuff, with a coupling adapted to connect to a syringe.
with a provision for a connector at its proximal end to be connected to a ventilator.

2 An endotracheal tube with aerosol delivery apparatus
with provision for aerosol delivery of medications to the lungs via a metered dose inhaler (MDI)
with at least one secondary canalization
with provision for an adapter at its proximal end
with an adapter designed to fit the nozzle of MDI cannister at its proximal end
an adapter with a pinhole opening at it's distal end that marks the origin of a secondary cannula.
the secondary canalization with an ID small enough for the aerosol particles generated by a MDI at the proximal end to be delivered at the distal tip of the ET tube in aerosol form
the secondary cannulation with two parts—a semi-flexible or a semi-rigid structure outside the main tubular structure and a rigid track within the wall of the ET tube
the secondary canalization with only one opening at the distal tip of the ET tube without protrusion of the cannulation beyond the body of the ET tube
the secondary canalization with a track from the outer surface to the inner surface within the wall of the ET tube.
